(19) United States
(12) Patent Application Publication
(10) Pub. No.: US 2009/0023755 A1
(43) Pub. Date: Jan. 22, 2009

(54) USE OF ISTRADEFYLLINE FOR TREATING BEHAVIORAL DISORDERS

(75) Inventors: Shizuo Shiozaki, Fuji-shi (JP); Junichi Shimada, Mishima-shi (JP); Hiroshi Kase, Tokyo (JP); Mayumi Shindo, Dunedin (NZ)

Correspondence Address: FITZPATRICK CELLA HARPER & SCINTO, 30 ROCKEFELLER PLAZA, NEW YORK, NY, 10112, US

(73) Assignee: KYOWA HAKKO KOGYO CO., LTD., Tokyo (JP)

(21) Appl. No.: 12/239,955

(22) Filed: Sep. 29, 2008

Related U.S. Application Data
(63) Continuation of application No. 10/539,574, filed on Jun. 17, 2005.
(60) Provisional application No. 60/509,039, filed on Dec. 27, 2002.

(51) Int. Cl. A61K 31/522; A61P 25/20; A61P 25/14

(52) U.S. Cl. 514/263.34

(57) ABSTRACT

The present invention provides a method of treating behavioral disorders such as attention deficit hyperactivity disorder, comprising administering an effective amount of (E)-8-(3,4-dimethoxystyryl)-1,3-diethyl-7-methylxanthine or a pharmaceutically acceptable salt thereof to a patient in need thereof and the like.

### FIELD OF THE INVENTION

The present invention relates to a method of treating behavioral disorders such as attention deficit hyperactivity disorder.

### BACKGROUND OF THE INVENTION

Attention deficit hyperactivity disorder (“ADHD”) is a behavioral disorder commonly diagnosed in childhood, estimated to affect 2 to 9.5 percent of all school-age children worldwide. One half to two thirds of these children will continue to suffer into adulthood. Its core symptoms include developmentally inappropriate levels of attention, concentration, activity, distractibility, and impulsivity. ADHD is thus characterized by hyperactive motor behavior, decreased attention span, impulsiveness and a variety of cognitive and perceptual problems. Children with ADHD usually have functional impairment across multiple settings including home, school, and peer relationships. ADHD has also been shown to have long-term adverse effects on academic performance, vocational success, and social-emotional development.

The direct and immediate causes of ADHD have not been known yet. Neurological imaging studies suggest involvement of the prefrontal cortex, part of the cerebellum, and at least two of the clusters of nerve cells deep in the brain that are collectively known as the basal ganglia. The right prefrontal cortex, two basal ganglia called the caudate nucleus and the globus pallidus, and the vermis region of the cerebellum were found to be significantly smaller than normal in children with ADHD (Scientific American, pp. 66-71, September 1998). The brain areas that are reduced in size in children with ADHD are the very ones that regulate attention. Genetics can contribute to ADHD. ADHD risk of a child whose identical twin has the disorder is 11 to 18 times greater than that of a nontwin sibling of a child with ADHD. Mutations in several genes that are normally very active in the prefrontal cortex and basal ganglia have been suggested to play a role in structural shrinking of the brain areas in ADHD. Particular variations in dopamine transporter gene, DAT 1, and dopamine receptor gene D4 were found more likely in children with ADHD (Scientific American, pp. 66-71, September 1998). Adenosine A_{2A} receptor polymorphisms have also been reported in ADHD [Clinical Genetics, 58, pp. 31-40 (2000)].

Despite progress in the assessment, diagnosis, and treatment of children and adults with ADHD, the disorder has remained controversial. One of the major controversies regarding ADHD concerns the use of psychostimulants to treat the condition. Psychostimulants, including amphetamine, methylphenidate, and pemoline, are by far the most widely researched and commonly prescribed treatments for ADHD [National Institutes of Health Consensus Development Conference Statement 1998 Nov. 16-18; 16(2): 1-37]. Because psychostimulants are more readily available and are being prescribed more frequently, concerns have intensified over their potential overuse and abuse. Very high doses of psychostimulants, particularly of amphetamines, may cause central nervous system damage, cardiovascular damage, and hypertension. In addition, high doses have been associated with compulsive behaviors and, in certain vulnerable individuals, movement disorders. There is a rare percentage of children and adults treated at high doses who have hallucinogenic responses. Drugs used for ADHD other than psychostimulants have their own adverse reactions: tricyclic antidepressants may induce cardiac arrhythmias, bupropion at high doses can cause seizures, and pemoline is associated with liver damage [National Institutes of Health Consensus Development Conference Statement 1998 Nov. 16-18; 16(2): 1-37]. Thus, efficacious and safer prophylactic or therapeutic agents of ADHD are needed.

Tic/Tourette's disorder is described in the Diagnostic and Statistical Manual of Mental Disorders (Fourth Edition—Revised, 1994, published by the American Psychiatric Association, Washington, D.C., U.S.A., pp. 100-105). Tic/Tourette's disorder is a behavioral disorder commonly diagnosed in childhood or adolescence, estimated to affect 4 to 5 individuals per 10,000, and it is reported that this disorder is approximately 1.5 to 3 times more common in males than in females. The following four disorders are included in Tic/Tourette's disorder: Tourette's disorder, chronic motor or vocal tic disorder, transient tic disorder, and tic disorder not otherwise specified.

A tic is a sudden, rapid, recurrent, nonrythmic, stereotyped motor movement or vocalization, and the symptoms are irresistible but can be suppressed after a lapse of time. All forms of tics may be exacerbated by stress and attenuated during absorbing activities.

The essential features of Tourette's disorder are multiple motor tics and one or more vocal tics. These features may appear simultaneously or separately.

The age at the onset of Tourette's disorder may be as early as age 2, is usually during childhood or early adolescence, and is by definition before age 18. The median age at the onset of motor tic is 7 years. The duration of the disorder is usually lifelong, though periods of remission lasting from weeks to years may occur. In most cases, the severity, frequency, and variability of the symptoms diminish during adolescence and adulthood. In other cases, the symptoms disappear entirely, usually by early adulthood.

Frequently comorbid with Tourette's disorder, ADHD has prevalence of 20-90 percent within clinic populations (Kaplan & Sadock's Comprehensive Textbook of Psychiatry, seventh edition, 2000, Lippincott Williams & Wilkins, Philadelphia).

The vulnerability to Tourette's disorder and related disorders is transmitted in an autosomal dominant pattern.

The major form of treatment of Tic/Tourette's disorder continues to be based on high-potency “typical” neuroleptics (tiaprid, pimozide, haloperidol, and the like), which may induce a wide range of potentially serious side effects.

WO 99/12546 discloses that some xanthine derivatives have an inhibitory action on neurodegeneration and are useful as a therapeutic agent for neurodegenerative disorders such as Alzheimer's disease, progressive supranuclear palsy, AIDS brain fever, propagating spongy brain fever, Huntington's chorea, multiple sclerosis, amyotrophic lateral sclerosis (ALS), multi-system atrophy, brain ischemia, and attention deficit hyperactivity disorder.

### SUMMARY OF THE INVENTION

The object of the present invention is to provide an excellent method of treating behavioral disorders such as attention deficit hyperactivity disorder.

### BRIEF DESCRIPTION OF THE DRAWINGS

FIG. 1 is a graph showing the effect of Compound (I) on locomotor activity in 6-hydroxydopamine-treated or vehicle-treated rats. *means P<0.05 compared with vehicle-treated rats. CI means Compound (I).

### DETAILED DESCRIPTION OF THE INVENTION

The present invention relates to the following (1) to (9).

(1) A method of treating a behavioral disorder, comprising administering an effective amount of (E)-8-(3,4-dimethoxystyryl)-1,3-diethyl-7-methylxanthine [hereinafter referred to as Compound (I)] or a pharmaceutically acceptable salt thereof to a patient in need thereof.
(2) Use of Compound (I) or a pharmaceutically acceptable salt thereof for manufacturing a therapeutic agent for the treatment of a behavioral disorder.
(3) A therapeutic agent for a behavioral disorder comprising Compound (I) or a pharmaceutically acceptable salt thereof.
(4) The method of treating a behavioral disorder according to the above (1), wherein the behavioral disorder is attention deficit hyperactivity disorder.
(5) The use according to the above (2), wherein the behavioral disorder is attention deficit hyperactivity disorder.
(6) The therapeutic agent for a behavioral disorder according to the above (3), wherein the behavioral disorder is attention deficit hyperactivity disorder.
(7) The method of treating a behavioral disorder according to the above (1), wherein the behavioral disorder is Tic/Tourette's disorder.
(8) The use according to the above (2), wherein the behavioral disorder is Tic/Tourette's disorder.
(9) The therapeutic agent for a behavioral disorder according to the above (3), wherein the behavioral disorder is Tic/Tourette's disorder.

Tic/Tourette's disorder includes Tourette's disorder, chronic motor or vocal tic disorder, transient tic disorder, and tic disorder not otherwise specified.

The pharmaceutically acceptable salts of Compound (I) include pharmaceutically acceptable acid addition salts, metal salts, ammonium salts, organic amine addition salts and amino acid addition salts.

The pharmaceutically acceptable acid addition salts of Compound (I) include inorganic acid addition salts such as hydrochloride, sulfate and phosphate, and organic acid addition salts such as acetate, maleate, fumarate, tartrate, citrate and methanesulfonate; the pharmaceutically acceptable metal salts include alkali metal salts such as sodium salt and potassium salt, alkaline earth metal salts such as magnesium salt and calcium salt, aluminum salt, and zinc salt; the pharmaceutically acceptable ammonium salts include ammonium and tetramethylammonium; the pharmaceutically acceptable organic amine addition salts include salts with morpholine and piperidine; and the pharmaceutically acceptable amino acid addition salts include salts with lysine, glycine and phenylalanine.

Compound (I) can be produced by the method disclosed in Japanese Published Unexamined Patent Application No. 211856/94, Japanese Published Unexamined Patent Application No. 16559/94 or WO 94/01114, or according to these methods. The desired compound in the process can be isolated and purified by purification methods conventionally used in synthetic organic chemistry, such as filtration, extraction, washing, drying, concentration, recrystallization or various kinds of chromatography.

In the case where a salt of compound (I) is desired and it is produced in the form of a desired salt, it may be subjected to purification as such. In the case where compound (I) is produced in the free form and its salt is desired, it is dissolved or suspended in a suitable solvent, and then an acid or a base may be added thereto to form the salt.

Compound (I) and pharmaceutically acceptable salts thereof may be in the form of adducts with water or various solvents, which can satisfactorily be used in the method or the use, or as the therapeutic agent of the present invention.

The physicochemical data of Compound (I) are described below. Compound 1:

Melting point: 190.4-191.3° C.

Elemental analysis: C₂₀H₂₄N₄O₄

Calcd. (%): C, 62.48; H, 6.29; N, 14.57

Found (%): C, 62.52; H, 6.53; N, 14.56

IR(KBr) νmax(cm⁻¹): 1697, 1655, 1518

NMR (CDCl₃, 270 MHz) δ(ppm): 7.74 (1H, d, J=15.5 Hz), 7.18 (1H, dd, J=8.3, 1.9 Hz), 7.08 (1H, d, J=1.9 Hz), 6.89 (1H, d, J=8.3 Hz), 6.77 (1H, d, J=15.5 Hz), 4.21 (2H, q, J=6.9 Hz), 4.09 (2H, q, J=6.9 Hz), 4.06 (3H, s), 3.96 (3H, s), 3.93 (3H, s), 1.39 (3H, t, J=6.9 Hz), 1.27 (3H, t, J=6.9 Hz)

A striking feature of ADHD is the unusual response to stimulant medication. Thus, administration of amphetamine to children with ADHD results in a sharp decrease in motor activity. Since the usual pharmacological response to amphetamine is an increase in motor activity, this response has been termed “paradoxical”. In rat pups treated with 6-hydroxydopamine, administration of methamphetamine reduces the hyperactivity, an effect paralleling the paradoxical response to the agent in ADHD. Accordingly, 6-hydroxydopamine-treated rat pups are an accepted model for ADHD in humans [Nature, 264, pp. 153-155 (1976)].

The pharmacological actions of Compound (I) are described in test examples.

### TEST EXAMPLE 1

### Effect of Compound (I) on Locomotor Activity in 6-Hydroxydopamine-Treated Neonatal Rats

Methods: Female neonatal SD rats were used for the experiments. 100 μg 6-Hydroxydopamine (6-HODA) was dissolved in a 0.1% solution of ascorbic acid in saline, and the obtained solution or 0.1% ascorbic acid in saline (control) was injected first at 3 days of age into the left lateral ventricle of the rat and secondly at 6 days of age into the right lateral ventricle of the rat. At 30-37 days, locomotor activity was measured by placing the rat in a transparent acrylic box (50×50×50 cm) 60 minutes after the drug administration using digital counters with infrared sensors (Scanet MV-10MT; Toyo Sangyo Co. Ltd., Toyama, Japan).

Compound (I) was suspended in a 0.3% aqueous Tween 80 solution, and administered orally to 6-HODA treated rats.

Results: The intracerebroventricular administrations of 6-HODA to pups resulted in increase of locomotor activity compared with vehicle treatment control. Compound (I), administered orally at 1.25 mg/kg and 5 mg/kg to 6-HODA treated rats, decreased locomotor activities, whereas it increased locomotor activities of control rats treated only with 0.1% ascorbic acid in saline, vehicle.

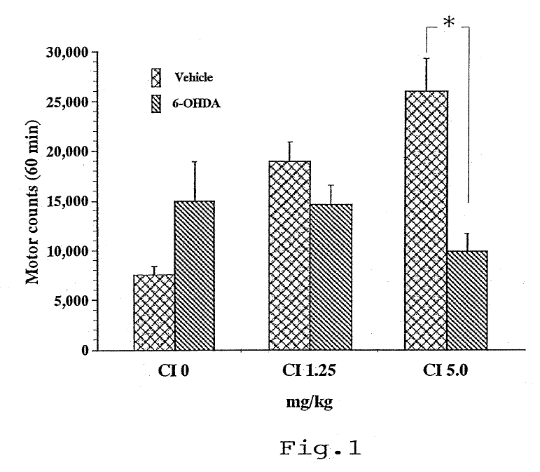

The results are shown in FIG. 1.

The above results indicate that Compound (I) is effective for improving ADHD.

### TEST EXAMPLE 2

### Effect of Compound (I) on Tic/Tourette Like Symptoms in 6-Hydroxydopamine-Treated Young Rats

Methods: 6-HODA was injected into the left medial forebrain bundle of a rat to induce a unilateral lesion of dopaminergic neurons, followed by repeated oral administration of L-DOPA at 20 mg/kg twice daily for 2 weeks to make a rat model of tic-like symptoms.

Tic-like, abnormal involuntary movements were observed after day 3 during repetitive treatment with L-DOPA. Two subtypes of involuntary movements were classified as axial (lateral torsion of the head, neck and trunk towards the side contralateral to the lesion, including swing of the head) and forelimb (abnormal movements contralateral to the lesion, including kicking movements of the forelimb).

The severity of these movements was assigned a score from 0 to 4 to each movement as follows.

### Axial

(score 0) no deviation of head

(score 1) lateral deviation of head: 300 or less

(score 2) lateral deviation of head: more than 30°, and 60° or less

(score 3) torsion of head and upper trunk: more than 60°, and 90° or less

(score 4) torsion of head and trunk: more than 90°

### Forelimb

(score 0) no movements of both distal and proximal forelimbs

(score 1) tiny oscillatory movements of the distal forelimb

(score 2) movements of low amplitude but causing visible translocation of both distal and proximal forelimbs

(score 3) translocation of the whole limb with visible contraction of shoulder muscles

(score 4) vigorous limb and shoulder movements of maximal amplitude

Compound (I) was repeatedly administered orally to 6-HODA treated rats at 1 mg/kg for 23 days, and Tic-like, abnormal involuntary movements were observed every 10 minutes for 3 hours, each time for one minute.

Peak score was obtained by adding the peak score for forelimb to that for axial (Data are expressed as mean±standard deviation in the following Table 1). Peak time means the time after the first administration when peak score was score observed.

Results: Compound (I), administered orally at 1 mg/kg, decreased peak score and peak time compared with those before administration of Compound (I).

The results are shown in Table 1.

TABLE 1
EFFECTS OF SUBSTANCE OH TIC/TOURETTE LIKE SYMPTOMS
IN 6-HYDROXYDOPAMINE-TREATED YOUNG RATS

| | | |
|---|---|---|
| Treatment | | |
| (mg/kg) | peak score | |
| p.o. -60 min | (n = 6) | peak time (min) |

| Pre-treatment | | 7.0 ± 1.0 | 110 |
|---|---|---|---|
| L-DOPA + | Day 1 | 5.6 ± 1.4 | 30 |
| Compound (1) | Day 9 | 4.4 ± 1.6 | 40 |
| (1 mg/kg) | Day 23 | 1.6 ± 0.6 | 20 |

The above results indicate that Compound (I) is effective for improving Tic/Tourette's disorder.

### TEST EXAMPLE 3

### Effect of Compound (I) on the Acquisition of a Delayed Alternation Task in the Young Rats

The following experiment was carried out according to a method described in Drug Dev. Res., 35, p. 83-95 (1996) with a slight modification. Methods: Male Rj: Wistar (Han) rats were used for the experiments. Before being tested, the rats were given the standard diet each day. Several 45 mg food pellets (these were also used in the delayed alternation sessions described below) were also given them to habituate them to this novel food.

The aim of this phase is to train rats, on the presentation of a single centralized retractable lever, to press on it to receive a food pellet.

The rats were subjected to 10 lever-pressing acquisition sessions in the experimental chambers according to a fixed ratio (FR1) schedule of reinforcement. Reinforcement consists of food pellets (45 mg) delivered after each lever-press. Each daily session lasts 15 minutes. All rats received an intraperitoneal administration of physiological saline 30 minutes before each session. During the first 7 sessions, the Skinner boxes were equipped with only one fixed lever situated centrally above the food receptacle, to avoid spatial preference for the right or the left side of the experimental panel. After the 7th lever-pressing session, the boxes were equipped with two retractable levers located on either side of the food receptacle. The rats were then subjected to 3 consecutive sessions in which the left or the right lever was pseudo-randomly presented every 5 seconds. At the end of this phase 80 to 100% of the rats acquired the lever press-response. Rats which failed to learn were excluded from the experiments. If some rats were close to establishing steady lever-pressing behavior they were given extra training with the aim of attaining at least 10 rats per group. Rats were assigned to treatment groups matched on the basis of their performance.

Subsequent to lever-press acquisition sessions, all rats were subjected to delayed alternation sessions. The test was conducted for 5 days. During this phase, the boxes were equipped with two retractable levers on each side of the food distributor. Each session consisted of 35 successive trials every 10 seconds. In each trial, the rat was first presented with one lever (left or right). When the rat pressed on the lever, the rat was given a food pellet, the lever was retracted and 5 seconds later two levers were presented. The rat had to learn to press on the lever opposite to that previously presented to gain a food pellet (non-matching to sample). If the rat did not respond to a one- or two-lever presentation within 20 seconds, the lever(s) were withdrawn and the next trial commenced 10 seconds later.

Compound (I) was suspended in 0.5% methylcellulose in distilled water and administered orally 60 minutes before each session.

The effect of Compound (I) was evaluated by measuring simple reaction time, which means the reaction time to each one-lever presentation, and choice reaction time, which means the reaction time to each two-lever presentation.

### Results:

### (Simple Reaction Time)

Compound (I), administered orally at 0.3 mg/kg, significantly decreased simple reaction times compared with those obtained in control rats treated only with 0.5% methylcellulose, vehicle.

The results are shown in Table 2-A.

TABLE 2-A
EFFECTS Of SUBSTANCE ON SIMPLE REACTION TIMES OF YOUNG
RATS IN THE DELAYED ALTERNATION ACQUISITION TEST

| Treatment | Simple reaction times per session (seconds) | | | | |
|---|---|---|---|---|---|
| (mg/kg) | (mean ± s.e.m.) | | | | |
| p.o. -60 min | S1 | S2 | S3 | S4 | S5 |
| Vehicle | 4.56 ± 0.34 | 3.53 ± 0.29 | 2.68 ± 0.28 | 2.06 ± 0.20 | 2.01 ± 0.22 |
| KW-6002 | 3.16 ± 0.24 | 2.49 ± 0.28 | 1.63 ± 0.20 | 1.73 ± 0.21 | 1.33 ± 0.11 |
| 0.3 | ** | * | * | NS | * |

Student's t test:
NS = Not Significant;
* = p < 0.05;
** = p < 0.01

The above results indicate that Compound (I) is effective for improving ADHD.

### (Choice Reaction Time)

Compound (I), administered orally at 0.3 mg/kg, significantly decreased choice reaction times compared with those obtained in control rats treated only with 0.5% methylcellulose, vehicle.

The results are shown in Table 2-B.

TABLE 2-B
EFFECTS OF SUBSTANCE ON CHOICE REACTION TIMES OF YOUNG
RATS IN THE DELAYED ALTERNATION ACQUISITION TEST

| Treatment | Choice reaction times per session (seconds) | | | | |
|---|---|---|---|---|---|
| (mg/kg) | (mean ± s.e.m.) | | | | |
| p.o. -60 min | S1 | S2 | S3 | S4 | S5 |
| Vehicle | 2.26 ± 0.22 | 1.99 ± 0.20 | 1.57 ± 0.19 | 1.16 ± 0.12 | 1.27 ± 0.16 |
| KW-6002 | 1.71 ± 0.13 | 1.52 ± 0.10 | 1.22 ± 0.12 | 1.13 ± 0.18 | 0.92 ± 0.09 |
| 0.3 | * | * | NS | NS | NS |

Student's t test:
NS = Not Significant;
* = p < 0.05

The above results indicate that Compound (I) is effective for improving ADHD.

### TEST EXAMPLE 4

### Acute Toxicity Test

Compound (I) was orally or intraperitoneally administered to groups of dd-strain male mice weighing 20±1 g, each group consisting of three mice. Seven days after the administration, the mortality was observed to determine a minimum lethal dose (MLD) of Compound (I).

The MLD value of Compound (I) was greater than 1000 mg/kg for oral administration.

Compound (I) or pharmaceutically acceptable salts thereof can be used as such or in the form of various pharmaceutical compositions. The pharmaceutical compositions of the present invention can be prepared by uniformly mixing an effective amount of compound (I) or a pharmaceutically acceptable salt thereof as an active ingredient with pharmaceutically acceptable carriers. The pharmaceutical compositions are preferably in a unit dosage form suitable for rectal administration, oral or parenteral (including subcutaneous, intravenous and intramuscular administration) administration, etc.

For preparing a pharmaceutical composition for oral administration, any useful pharmaceutically acceptable carriers can be used. For example, liquid preparations for oral administration such as suspension and syrup can be prepared using water; sugars such as sucrose, sorbitol and fructose; glycols such as polyethylene glycol and propylene glycol; oils such as sesame oil, olive oil and soybean oil; preservatives such as a p-hydroxybenzoate; flavors such as strawberry flavor and peppermint, etc. Powder, pills, capsules and tablets can be prepared using excipients such as lactose, glucose, sucrose and mannitol; disintegrating agents such as starch and sodium alginate; lubricants such as magnesium stearate and talc; binders such as polyvinyl alcohol, hydroxypropyl cellulose and gelatin; surfactants such as fatty acid esters; plasticizers such as glycerin, etc. Tablets and capsules are the most useful oral unit dosage because of the readiness of administration. For preparing tablets and capsules, solid pharmaceutical carriers are used.

Injectable preparations can be prepared using carriers such as distilled water, a salt solution, a glucose solution and a mixture of a salt solution and a glucose solution. The preparation can be prepared in the form of solution, suspension or dispersion according to a conventional method by using a suitable auxiliary.

Compound (I) or a pharmaceutically acceptable salt thereof can be administered orally in the pharmaceutical form described above or parenterally as the injection. The effective dose and administration schedule vary depending on the mode of administration, age, weight, and symptoms of a patient, etc. However, generally, compound (I) or a pharmaceutically acceptable salt thereof is administered in a dose of 1 to 900 mg/60 kg/day, preferably in a dose of 1 to 200 mg/60 kg/day.

Certain embodiments of the present invention are described in the following examples.

### EXAMPLE 1

### Tablets

Tablets having the following composition were prepared in a conventional manner.

Compound (I) (40 g) was mixed with 286.8 g of lactose and 60 g of potato starch, followed by addition of 120 g of a 10% aqueous solution of hydroxypropyl cellulose. The resultant mixture was kneaded, granulated, and then dried by a conventional method. The granules were refined to give granules used to make tablets. After mixing the granules with 1.2 g of magnesium stearate, the mixture was formed into tablets each containing 20 mg of the active ingredient by using a tablet maker (Model RT-15, Kikusui) having pestles of 8 mm diameter.

The prescription is shown in Table 3.

TABLE 3

| | | |
|---|---|---|
| Compound (I) | 20 | mg |
| Lactose. | 143.4 | mg |
| Potato Starch | 30 | mg |
| Hydroxypropyl Cellulose | 6 | mg |
| Magnesium Stearate | 0.6 | mg |
| | 200 | mg |

### EXAMPLE 2

### Capsules

Capsules having the following composition were prepared in a conventional manner.

Compound (I) (200 g) was mixed with 995 g of Avicel and 5 g of magnesium stearate. The mixture was put in hard capsules No. 4 each having a capacity of 120 mg by using a capsule filler (Model LZ-64, Zanashi) to give capsules each containing 20 mg of the active ingredient.

The prescription is shown in Table 4.

TABLE 4

| | | |
|---|---|---|
| Compound (I) | 20 | mg |
| Avicel | 99.5 | mg |
| Magnesium Stearate | 0.5 | mg |
| | 120 | mg |

### EXAMPLE 3

### Injections

Injections having the following composition were prepared in a conventional manner.

Compound (I) (1 g) was dissolved in 100 g of purified soybean oil, followed by addition of 12 g of purified egg yolk lecithin and 25 g of glycerin for injection. The resultant mixture was made up to 1,000 ml with distilled water for injection, thoroughly mixed, and emulsified by a conventional method. The resultant dispersion was subjected to aseptic filtration by using 0.2 μm disposable membrane filters, and then aseptically put into glass vials in 2 ml portions to give injections containing 2 mg of the active ingredient per vial.

The prescription is shown in Table 5.

TABLE 5

| | | |
|---|---|---|
| Compound (I) | 2 | mg |
| Purified Soybean Oil | 200 | mg |
| Purified Egg Yolk Lecithin | 24 | mg |
| Glycerine for Injection | 50 | mg |
| Distilled Water for Injection | 1.72 | ml |
| | 2.00 | ml |

1. A method of treating attention deficit hyperactivity disorder, comprising administering an effective amount of (E)-8-(3,4-dimethoxystyryl)-1,3-diethyl-7-methylxanthine, or a pharmaceutically acceptable salt thereof, to a patient in need thereof.

2-6. (canceled)

7. A method of treating Tic/Tourette's disorder, comprising administering an effective amount of (E)-8-(3,4-dimethoxystyryl)-1,3-diethyl-7-methylxanthine, or a pharmaceutically acceptable salt thereof, to a patient in need thereof.

8-9. (canceled)
